(19) United States
(12) Patent Application Publication
(10) Pub. No.: US 2005/0081852 A1
(43) Pub. Date: Apr. 21, 2005

(54) Package for an aerosolization apparatus and pharmaceutical formulation receptacle

(75) Inventor: Narasimhan Rangachari, Cupertino, CA (US)

Correspondence Address: NEKTAR THERAPEUTICS, 150 INDUSTRIAL ROAD, SAN CARLOS, CA, 94070, US

(73) Assignee: Nektar Therapeutics (formely Inhale Therapeutic System Inc.), San Carlos, CA (US)

(21) Appl. No.: 10/744,910

(22) Filed: Dec. 22, 2003

Related U.S. Application Data
(60) Provisional application No. 60/437,339, filed on Dec. 30, 2002.

(51) Int. Cl. A61M 15/00; A61M 16/00

(52) U.S. Cl. 128/203.21

(57) ABSTRACT

A pharmaceutical package comprises a first compartment and a second compartment. The first compartment contains an aerosolization apparatus, the aerosolization apparatus comprising a housing defining a chamber having one or more air inlets and one or more air outlets, the chamber being sized to contain a capsule; a puncturing mechanism moveable within the housing to create one or more openings in a capsule in the chamber; and a mouth or nose piece in communication with the one or more air outlets, whereby when a user inhales, air enters into the chamber through the inlet to cause the capsule to move within the chamber so that the pharmaceutical formulation exits through one or more openings in the capsule and is aerosolized and transported through the outlet for delivery to the user through the mouth or nose piece. The second compartment contains a capsule. The capsule is positionable within the chamber of the aerosolization apparatus during use of the aerosolization apparatus and contains an aerosolizable powder pharmaceutical formulation comprising amphotericin B. The capsule is contained within the second compartment by a moisture barrier seal.

This application claims the benefit U.S. Provisional Patent Application Ser. No. 60/437,339 filed on Dec. 30, 2002, which is incorporated herein by reference in its entirety.

### BACKGROUND

The need for effective therapeutic treatment of patients has resulted in the development of a variety of pharmaceutical formulation delivery techniques. One traditional technique involves the oral delivery of a pharmaceutical formulation in the form of a pill, capsule, elixir, or the like. However, oral delivery can in some cases be undesirable. For example, many pharmaceutical formulations may be degraded in the digestive tract before they can be effectively absorbed by the body. Inhaleable drug delivery, where an aerosolized pharmaceutical formulation is orally or nasally inhaled by a patient to deliver the formulation to the patient's respiratory tract, has proven to be a particularly effective and/or desirable alternative. For example, in one inhalation technique, an aerosolized pharmaceutical formulation provides local therapeutic relief to a portion of the respiratory tract, such as the lungs, to treat diseases such as asthma, emphysema, and cystic fibrosis. In another inhalation technique, a pharmaceutical formulation is delivered deep within a patient's lungs where it may be absorbed into the blood stream. Many types of inhalation devices exist including devices that aerosolize a dry powder pharmaceutical formulation.

In one dry powder aerosolization technique, a receptacle, such as a capsule or a blister pack, containing an inhaleable dry powder is loaded into a chamber in an aerosolization apparatus. Within the chamber, the dry powder is at least partially emptied and dispersed to aerosolize the dry powder so that it may be inhaled by a patient. However, in conventional devices, the separate packaging of the apparatus and the receptacle causes inconvenience for the user and/or for a pharmacist.

Therefore, it is desirable to be able to provide a package that is more convenient for users and for pharmacists. It is further desirable to provide a package that reduces the loss of a pharmaceutical formulation and that reduces the likelihood of using an aerosolization apparatus beyond its useful life.

### SUMMARY

The present invention satisfies these needs. In one aspect of the invention, a package is provided which simplifies the use of a pharmaceutical formulation for a user and/or for a pharmacist.

In another aspect of the invention, a pharmaceutical package comprises a first compartment and a second compartment. The first compartment contains an aerosolization apparatus, the aerosolization apparatus comprising a housing defining a chamber having one or more air inlets and one or more air outlets, the chamber being sized to contain a capsule; a puncturing mechanism moveable within the housing to create one or more openings in a capsule in the chamber; and a mouth or nose piece in communication with the one or more air outlets, whereby when a user inhales, air enters into the chamber through the inlet to cause the capsule to move within the chamber so that the pharmaceutical formulation exits through one or more openings in the capsule and is aerosolized and transported through the outlet for delivery to the user through the mouth or nose piece. The second compartment contains a capsule. The capsule is positionable within the chamber of the aerosolization apparatus during use of the aerosolization apparatus and contains an aerosolizable powder pharmaceutical formulation comprising amphotericin B. The capsule is contained within the second compartment by a moisture barrier seal.

In another aspect of the invention, a method of packaging a pharmaceutical formulation comprises placing an aerosolization apparatus in a first compartment and placing a capsule that may be inserted into the aerosolization apparatus in a second compartment, the capsule containing a pharmaceutical formulation comprising amphotericin B.

### DRAWINGS

These features, aspects, and advantages of the present invention will become better understood with regard to the following description, appended claims, and accompanying drawings which illustrate exemplary features of the invention. However, it is to be understood that each of the features can be used in the invention in general, not merely in the context of the particular drawings, and the invention includes any combination of these features, where:

FIG. 1 is a schematic top view of a version of a package according to the invention;

FIG. 2A is a schematic sectional side view of a version of an aerosolization apparatus in an initial position;

FIG. 2B is a schematic sectional side view of the version of an aerosolization apparatus shown in FIG. 2A at the beginning of an aerosolization process;

FIG. 2C is a schematic sectional side view of the version of an aerosolization apparatus shown in FIG. 2A during an aerosolization process;

FIG. 3A is a schematic sectional side view of a version of an aerosolization apparatus in a rest position;

FIG. 3B is a schematic sectional side view of the version of an aerosolization apparatus shown in FIG. 3A just before capsule puncture;

FIG. 3C is a schematic sectional side view of the version of an aerosolization apparatus shown in FIG. 3A as the capsule is being punctured;

FIG. 3D is a schematic sectional side view of the version of an aerosolization apparatus shown in FIG. 3A just after capsule puncture;

FIG. 3E is a schematic sectional side view of the version of an aerosolization apparatus shown in FIG. 3A in use;

FIG. 4 is a schematic perspective view of another version of a package according to the invention; and

FIG. 5 is a schematic perspective view of another version of a package according to the invention.

### DESCRIPTION

The present invention relates to storing a pharmaceutical formulation. Although the process is illustrated in the context of packaging a dry powder pharmaceutical formulation receptacle and aerosolization apparatus, the present invention can be used in other processes and should not be limited to the examples provided herein.

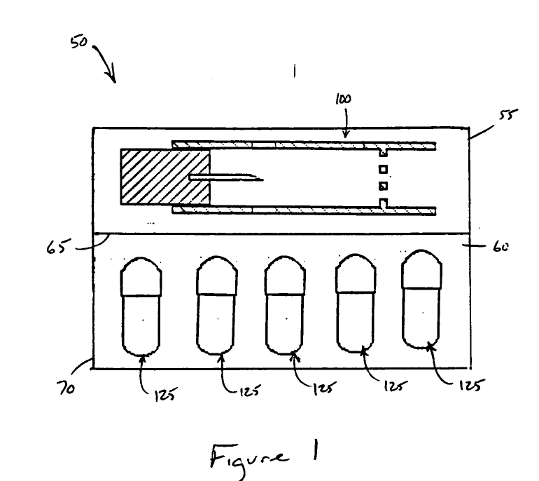

A package 50 according to the present invention is shown schematically in FIG. 1. The package 50 comprises a first compartment 55 and a second compartment 60. The first and second compartments are optionally separated by a wall or barrier 65. The first compartment is sized and shaped to securely receive and store an aerosolization apparatus 100. The second compartment is sized and shaped to securely store one or more receptacles 125 which contain a pharmaceutical formulation. The package 100 comprises a moisture barrier 70 that is adapted to provide protection against undesirable amounts of moisture coming in contact with the pharmaceutical formulation in the receptacles 125.

Some pharmaceutical formulations are particularly sensitive to moisture. For example, some dry powder pharmaceutical formulations that are to be aerosolized and inhaled by a user may become agglomerated when in the presence of excessive moisture. The agglomerations may affect the aerosol characteristics of the pharmaceutical formulation and reduce the therapeutic effects of the pharmaceutical formulation delivery. Accordingly, the package 100 of the present invention may be adapted to provide sufficient moisture protection over a predetermined amount of time for a particular pharmaceutical formulation. For example, the moisture barrier 115 or the combination of the moisture barrier 115 with the capsule 105 may provide moisture protection for at least about 2 days, more preferably for at least about 1 week, and most preferably for at least about 3 weeks. Examples of moisture protection packages are described in U.S. Provisional Patent Application Ser. No. 60/343,309 filed on Dec. 21, 2001, which is incorporated herein by reference in its entirety.

The moisture barrier 70 may be sufficiently thick to decrease the amount of moisture that is able to pass through the barrier 70. In one version, the moisture barrier 70 comprises a material that is resistant to moisture passage in order to reduce the thickness of the barrier 70. For example, the moisture barrier 70 may comprise one or more metals, such as aluminum or the like, and/or other moisture barrier materials, such as polyamides, polyvinyl chlorides and the like.

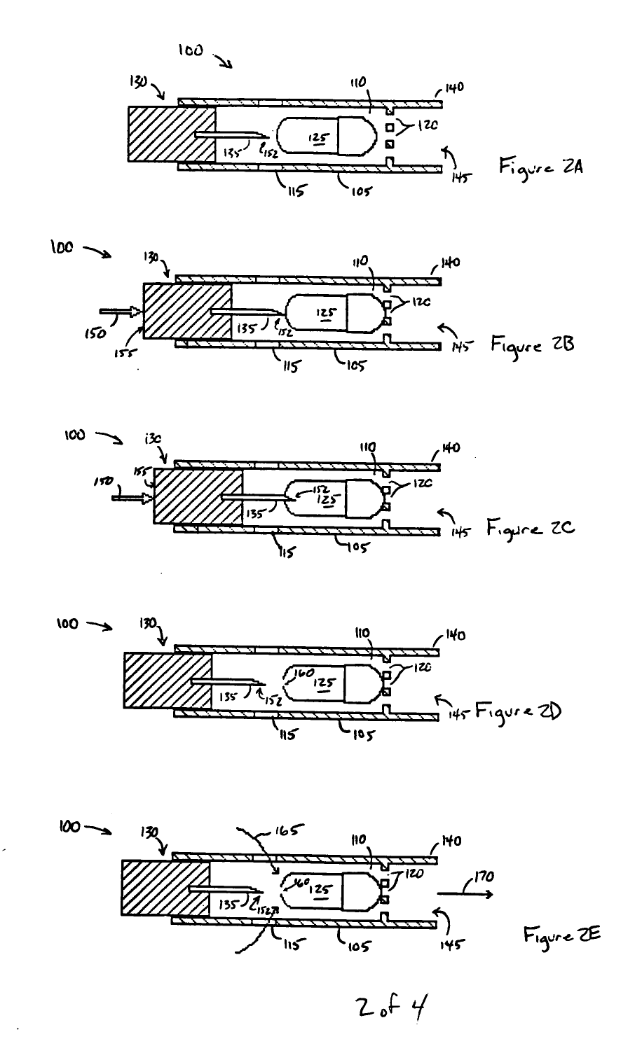

The aerosolization apparatus 100 is capable of aerosolizing the pharmaceutical formulation contained in the receptacle 125. An example of an aerosolization apparatus 100 is shown schematically in FIG. 2A. The aerosolization apparatus 100 comprises a housing 105 defining a chamber 110 having one or more air inlets 115 and one or more air outlets 120. The chamber 110 is sized to receive a receptacle 125 which contains an aerosolizable pharmaceutical formulation. A puncturing mechanism 130 comprises a puncture member 135 that is moveable within the chamber 110. Near or adjacent the outlet 120 is an end section 140 that may be sized and shaped to be received in a user's mouth or nose so that the user may inhale through an opening 145 in the end section 140 that is in communication with the outlet 120.

The aerosolization apparatus 100 utilizes air flowing through the chamber 110 to aerosolize the pharmaceutical formulation in the receptacle 125. For example, FIGS. 2A through 2E illustrate the operation of a version of an aerosolization apparatus 100 where air flowing through the inlet 115 is used to aerosolize the pharmaceutical formulation and the aerosolized pharmaceutical formulation flows through the outlet 120 so that it may be delivered to the user through the opening 145 in the end section 140. The aerosolization apparatus 100 is shown in its out-of-the-package or initial condition in FIG. 2A. A receptacle 125 is removed from the package 100 and is positioned within the chamber 110.

To use the aerosolization apparatus 100, the pharmaceutical formulation in the receptacle 125 is exposed to allow it to be aerosolized. In the version of FIGS. 2A though 2E, the puncture mechanism 130 is advanced within the chamber 110 by applying a force 150 to the puncture mechanism 130. For example, a user may press against a surface 155 of the puncturing mechanism 130 to cause the puncturing mechanism 130 to slide within the housing 105 so that the puncture member 135 contacts the receptacle 125 in the chamber 110, as shown in FIG. 2B. By continuing to apply the force 150, the puncture member 135 is advanced into and through the wall of the receptacle 125, as shown in FIG. 2C. The puncture member may comprise one or more sharpened tips 152 to facilitate the advancement through the wall of the receptacle 125. The puncturing mechanism 130 is then retracted to the position shown in FIG. 2D, leaving an opening 160 through the wall of the receptacle 125 to expose the pharmaceutical formulation in the receptacle 125.

Air or other gas then flows through an inlet 115, as shown by arrows 165 in FIG. 2E. The flow of air causes the pharmaceutical formulation to be aerosolized. When the user inhales 170 through the end section 140 the aerosolized pharmaceutical formulation is delivered to the user's respiratory tract. In one version, the air flow 165 may be caused by the user's inhalation 170. In another version, compressed air or other gas may be ejected into the inlet 115 to cause the aerosolizing air flow 165.

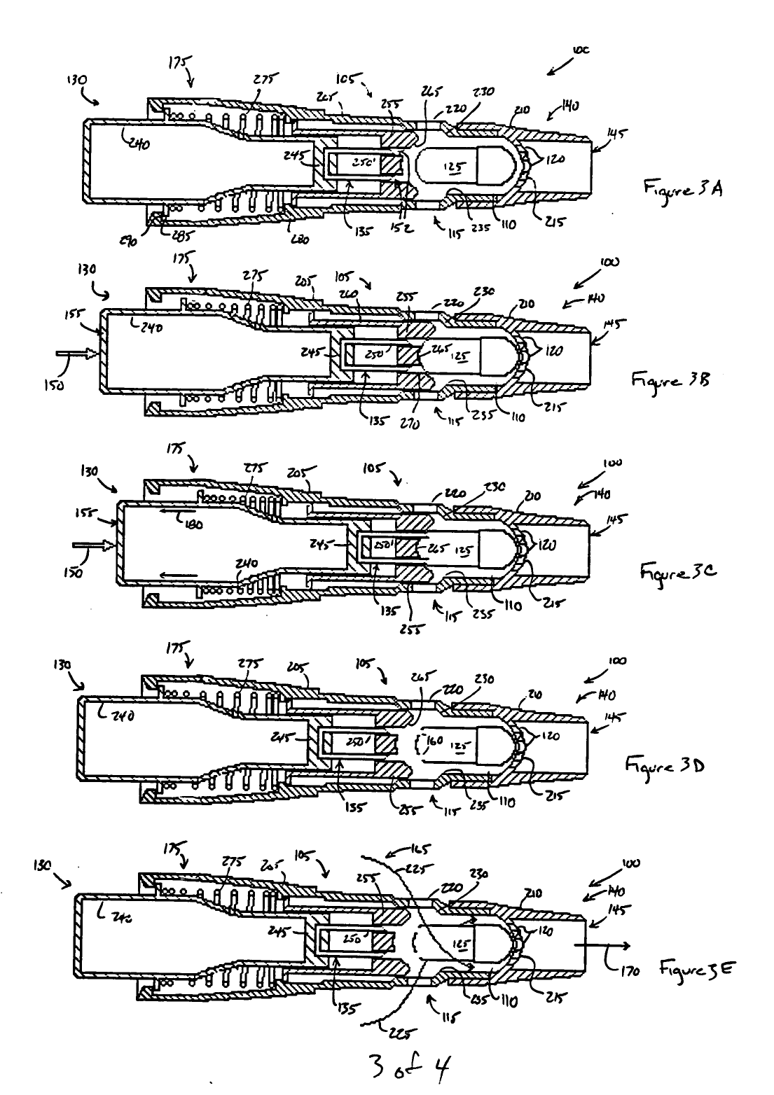

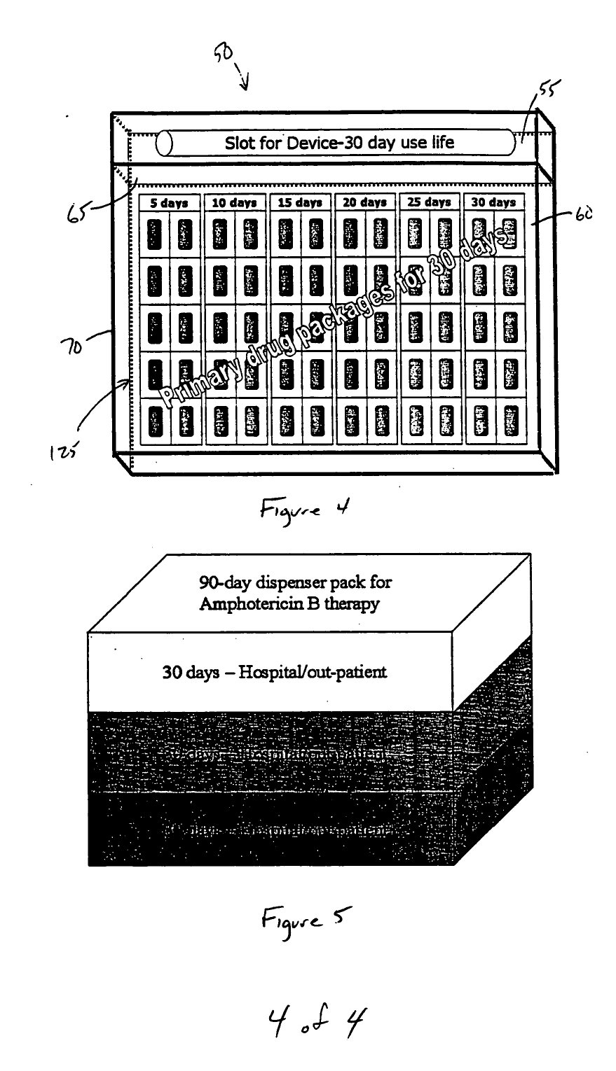

Another version of an aerosolization apparatus 100 is shown in FIGS. 3A through 3E. In this version, the housing 105 of the aerosolization apparatus 100 comprises a body 205 and a removable endpiece 210. The endpiece 210 may be removed from the body 205 to insert a receptacle 125 in the chamber 110 which is formed when the body 205 and the endpiece 210 are connected together. The endpiece 210 comprises a partition 215 that blocks the forward end of the chamber 110, and the partition 215 has the one or more outlets 120 extending therethrough. An example of an aerosolization apparatus with a partition 215 and chamber 110 are described in U.S. Pat. No. 4,069,819 and in U.S. Pat. No. 4,995,385, both of which are incorporated herein by reference in their entireties. In such an arrangement, the chamber 110 comprises a longitudinal axis that lies generally in the inhalation direction, and the receptacle 125 is insertable lengthwise into the chamber 110 so that the receptacle's longitudinal axis may be parallel to the longitudinal axis of the chamber 110. In the version of FIGS. 3A through 3E, the chamber 110 is sized to receive a receptacle 125 containing a pharmaceutical formulation in a manner which allows the receptacle to move within the chamber 110. The inlets 115 comprise a plurality of tangentially oriented slots 220. When a user inhales 170 through the endpiece 210, outside air is caused to flow through the tangential slots 220 as shown by arrows 225 in FIG. 3E. This airflow 225 creates a swirling airflow within the chamber 110. The swirling airflow causes the receptacle 125 to contact the partition 215 and then to move within the chamber 110 in a manner that causes the pharmaceutical formulation to exit the receptacle 125 and become entrained within the swirling airflow. In one version, the receptacle 125 may rotate within the chamber 110 in a manner where the longitudinal axis of the receptacle is remains at an angle less than 80 degrees, and preferably less than 45 degrees from the longitudinal axis of the chamber. The movement of the receptacle 125 in the chamber 110 may be caused by the width of the chamber 110 being less than the length of the receptacle 125. In one specific version, the chamber 110 comprises a tapered section 230 that terminates at an edge 235. During the flow of swirling air in the chamber 110, the forward end of the receptacle 125 contacts and rests on the partition 215 and a sidewall of the receptacle 125 contacts the edge 235 and slides and/or rotates along the edge 235. This motion of the receptacle is particularly effective in forcing a large amount of the pharmaceutical formulation through one or more openings 160 in the rear of the receptacle 125.

The one or more openings 160 in the rear of the receptacle 125 in the version of FIGS. 3A through 3E are created by a puncturing mechanism 130 that is slidable within the body 205. The puncturing mechanism 130, shown in its rest position in FIG. 3A, comprises a plunger 240 attached at its forward end 245 to the puncture member 135, which in the version shown is a U-shaped staple 250 having two sharpened tips 152. The puncturing mechanism 130 further comprises a seating member 255 which contacts the plunger 240 and/or the puncture member 135 and is slidable relative to the plunger 240 and the puncture member 135. To create the openings 160 in the receptacle 125, the user applies a force 150 to the plunger 240, as shown in FIG. 3B, such as by pressing against the end surface 155 of the plunger 240 with the user's finger or thumb. The force 150 causes the plunger to slide within the body 205. A slight frictional contact between the plunger 240 the a rear section 260 of the seating member 255 causes the seating member 255 to also slide within the body 205 until a forward seating surface 265 of the seating member 255 contacts the capsule 125, as shown in FIG. 3B. The forward seating surface 265, which may be shaped to generally match the shape of the receptacle 125, secures the receptacle 125 between the seating member 255 and the partition 215. The continued application of force 150 causes the plunger 240 and the puncture member 135 to slide relative to the seating member 255, as shown in FIG. 3C, to advance the puncture member 135 through openings 270 in the forward seating surface 265 and into the receptacle 125. Upon the removal of the force 150, a spring 275 or other biasing member urges the puncturing mechanism 130 back to its rest position. For example, the spring 275 may contact a shoulder 280 in the body 205 and press a flange 285 on the plunger 240 toward a rim 290 in the body 205. The frictional engagement between the plunger 240 and the seating member 255 also returns the seating member 255 to its retracted position when the plunger is returned to its retracted position.

In another version, the aerosolization apparatus 100 may be configured differently than as shown in FIGS. 2A through 2E and 3A through 3E. For example, the chamber 100 may be sized and shaped to receive the receptacle 125 so that the receptacle 125 is orthogonal to the inhalation direction, as described in U.S. Pat. No. 3,991,761. As also described in U.S. Pat. No. 3,991,761, the puncturing mechanism 130 may puncture both ends of the receptacle 125. In another version, the chamber may receive the receptacle 125 in a manner where air flows through the receptacle 125 as described for example in U.S. Pat. No. 4,338,931 and in U.S. Pat. No. 5,619,985. In another version, the aerosolization of the pharmaceutical formulation may be accomplished by pressurized gas flowing through the inlets, as described for example in U.S. Pat. No. 5,458,135, U.S. Pat. No. 5,785,049, and U.S. Pat. No. 6,257,233, or propellant, as described in PCT Publication WO 00/72904 and U.S. Pat. No. 4,114,615. All of the above references being incorporated herein by reference in their entireties.

In one version, the receptacle 125 comprises a capsule. The capsule may be of a suitable shape, size, and material to contain the pharmaceutical formulation and to provide the pharmaceutical formulation in a usable condition. For example, the capsule may comprise a wall which comprises a material that does not adversely react with the pharmaceutical formulation. In addition, the wall may comprise a material that allows the capsule to be opened to allow the pharmaceutical formulation to be aerosolized. In one version, the wall comprises one or more of gelatin, hydroxypropyl methylcellulose (HPMC), polyethyleneglycol-compounded HPMC, hydroxyproplycellulose, agar, or the like. Alternatively or additionally, the capsule wall may comprise a polymeric material, such as polyvinyl chloride (PVC). In one version, the capsule may comprise telescopically ajoined sections, as described for example in U.S. Pat. No. 4,247,066 which is incorporated herein by reference in its entirety. The interior of the capsule may be filled with a suitable amount of the pharmaceutical formulation, and the size of the capsule may be selected to adequately contain a desired amount of the pharmaceutical formulation. The sizes generally range from size 5 to size 000 with the outer diameters ranging from about 4.91 mm to 9.97 mm, the heights ranging from about 11.10 mm to about 26.14 mm, and the volumes ranging from about 0.13 ml to about 1.37 ml, respectively. Suitable capsules are available commercially from, for example, Shionogi Qualicaps Co. in Nara, Japan and Capsugel in Greenwood, S.C. After filling, a top portion may be placed over the bottom portion to form the a capsule shape and to contain the powder within the capsule, as described in U.S. Pat. No. 4,846,876, U.S. Pat. No. 6,357,490, and in the PCT application WO 00/07572 published on Feb. 17, 2000, all of which are incorporated herein by reference in their entireties.

In another version, the receptacle 125 may comprise package that contains the pharmaceutical formulation between stacked layers, such as a blister package. An example of a blister package is described in U.S. Provisional Patent Application 60/343,310, filed on Dec. 21, 2001, which is incorporated herein by reference in its entirety. The blister package may be used in an aerosolization apparatus 100 designed to aerosolize pharmaceutical formulation stored within a blister pack. For example, the aerosolization apparatus 100 may be of the type described in U.S. Pat. No. 6,257,233 and/or in PCT Application WO 01/00263, both of which are incorporated herein by reference in their entireties. In one version, the first compartment 55 may store a portion of an aerosolization apparatus, rather than the entire aerosolization apparatus. For example, the first compartment 55 may store a transjector, such as a transjector assembly described in U.S. Pat. No. 5,740,794 which is incorporated herein by reference in its entirety.

In one version, the package 50 contains sufficient supplies for a predetermined dosage regimen. For example, the package 50 may contain a single dose of pharmaceutical formulation. For some medicaments, an aerosolization apparatus must be operated two or more times, each time with a new receptacle 125, in order to administer a desired dose. Accordingly, in one version of the invention, the package 50 comprises an aerosolization apparatus 100 and the number of receptacles 125 necessary for administering the desired dose. The number of receptacles is dependent upon the size of the receptacles and the amount of pharmaceutical formulation filled into the receptacles. A pharmacist may prepare the package 50 for or administer the package 50 to a user in accordance with a physician's prescription. When the user needs to intake the medicament, the user need only open the package 50 and operate the aerosolization apparatus 100 for each of the receptacles 125 in the package 50. The empty package 50, the aerosolization apparatus 100 and the used receptacles 125 may then all be disposed of.

In another version, the package 50 contains an aerosolization apparatus 100 and a plurality of doses of receptacles 125. For example, the aerosolization apparatus 100 may have a certain usage life and the number of receptacles 125 may be an amount equal to the amount that would be used during the usage life. A version of such a package 50 is shown in FIG. 4. In this exemplary version, the aerosolization apparatus 100 has a usage life of 30 days. The receptacles 125 are provided so that two receptacles 125 per day may be used for the 30 days. In one version, the packaging of the receptacles may be in calendar format. As shown in FIG. 5, a plurality of the packages 50 may be packaged together to provide, for example, a 90 day supply of the medicament.

The design of the package 50 and/or the grouping of the packages 50 may be sufficient to cover an entire length of a therapy for a user. For example, the package 50 may be designed to cover a 30 day, or other period, one-time therapy. In one version, the package 50 may be prescribed to a patient undergoing anti-fungal therapy, such as Amphotericin B therapy. A typical dosing regimen for Amphotericin B therapy involves an initial dosing period of 90-100 days. The treatment is intended during the patient's neutropenic stage first in the hospital and then as an outpatient. The therapy may further be extended in some patients for an additional dosing period of 90 days either in or out of the hospital.

The package 50 of the present invention is advantageous in that it provides convenience to both the pharmacist and the user. The user does not need to make separate visits to the pharmacist for an aerosolization apparatus and for the pharmaceutical formulation. In addition, the user knows when to dispose of the device and is not tempted to over-use the aerosolization apparatus 100.

In a preferred version, the invention provides a system and method for aerosolizing a pharmaceutical formulation and delivering the pharmaceutical formulation to the respiratory tract of the user, and in particular to the lungs of the user. The pharmaceutical formulation may comprise powdered medicaments, liquid solutions or suspensions, and the like, and may include an active agent.

The active agent described herein includes an agent, drug, compound, composition of matter or mixture thereof which provides some pharmacologic, often beneficial, effect. This includes foods, food supplements, nutrients, drugs, vaccines, vitamins, and other beneficial agents. As used herein, the terms further include any physiologically or pharmacologically active substance that produces a localized or systemic effect in a patient. An active agent for incorporation in the pharmaceutical formulation described herein may be an inorganic or an organic compound, including, without limitation, drugs which act on: the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system, and the central nervous system. Suitable active agents may be selected from, for example, hypnotics and sedatives, psychic energizers, tranquilizers, respiratory drugs, anticonvulsants, muscle relaxants, antiparkinson agents (dopamine antagnonists), analgesics, anti-inflammatories, antianxiety drugs (anxiolytics), appetite suppressants, antimigraine agents, muscle contractants, anti-infectives (antibiotics, antivirals, antifungals, vaccines) antiarthritics, antimalarials, antiemetics, anepileptics, bronchodilators, cytokines, growth factors, anti-cancer agents, antithrombotic agents, antihypertensives, cardiovascular drugs, antiarrhythmics, antioxicants, anti-asthma agents, hormonal agents including contraceptives, sympathomimetics, diuretics, lipid regulating agents, antiandrogenic agents, antiparasitics, anticoagulants, neoplastics, antineoplastics, hypoglycemics, nutritional agents and supplements, growth supplements, antienteritis agents, vaccines, antibodies, diagnostic agents, and contrasting agents. The active agent, when administered by inhalation, may act locally or systemically.

The active agent may fall into one of a number of structural classes, including but not limited to small molecules, peptides, polypeptides, proteins, polysaccharides, steroids, proteins capable of eliciting physiological effects, nucleotides, oligonucleotides, polynucleotides, fats, electrolytes, and the like.

Examples of active agents suitable for use in this invention include but are not limited to one or more of calcitonin, erythropoietin (EPO), Factor VIII, Factor IX, ceredase, cerezyme, cyclosporin, granulocyte colony stimulating factor (GCSF), thrombopoietin (TPO), alpha-1 proteinase inhibitor, elcatonin, granulocyte macrophage colony stimulating factor (GMCSF), growth hormone, human growth hormone (HGH), growth hormone releasing hormone (GHRH), heparin, low molecular weight heparin (LMWH), interferon alpha, interferon beta, interferon gamma, interleukin-1 receptor, interleukin-2, interleukin-1 receptor antagonist, interleukin-3, interleukin-4, interleukin-6, luteinizing hormone releasing hormone (LHRH), factor IX, insulin, pro-insulin, insulin analogues (e.g., mono-acylated insulin as described in U.S. Pat. No. 5,922,675, which is incorporated herein by reference in its entirety), amylin, C-peptide, somatostatin, somatostatin analogs including octreotide, vasopressin, follicle stimulating hormone (FSH), insulin-like growth factor (IGF), insulintropin, macrophage colony stimulating factor (M-CSF), nerve growth factor (NGF), tissue growth factors, keratinocyte growth factor (KGF), glial growth factor (GGF), tumor necrosis factor (TNF), endothelial growth factors, parathyroid hormone (PTH), glucagon-like peptide thymosin alpha 1, IIb/IIIa inhibitor, alpha-1 antitrypsin, phosphodiesterase (PDE) compounds, VLA-4 inhibitors, bisphosponates, respiratory syncytial virus antibody, cystic fibrosis transmembrane regulator (CFTR) gene, deoxyreibonuclease (Dnase), bactericidal/permeability increasing protein (BPI), anti-CMV antibody, 13-cis retinoic acid, macrolides such as erythromycin, oleandomycin, troleandomycin, roxithromycin, clarithromycin, davercin, azithromycin, flurithromycin, dirithromycin, josamycin, spiromycin, midecamycin, leucomycin, miocamycin, rokitamycin, andazithromycin, and swinolide A; fluoroquinolones such as ciprofloxacin, ofloxacin, levofloxacin, trovafloxacin, alatrofloxacin, moxifloxicin, norfloxacin, enoxacin, grepafloxacin, gatifloxacin, lomefloxacin, sparfloxacin, temafloxacin, pefloxacin, amifloxacin, fleroxacin, tosufloxacin, prulifloxacin, irloxacin, pazufloxacin, clinafloxacin, and sitafloxacin, aminoglycosides such as gentamicin, netilmicin, paramecin, tobramycin, amikacin, kanamycin, neomycin, and streptomycin, vancomycin, teicoplanin, rampolanin, mideplanin, colistin, daptomycin, gramicidin, colistimethate, polymixins such as polymixin B, capreomycin, bacitracin, penems; penicillins including penicllinase-sensitive agents like penicillin G, penicillin V, penicillinase-resistant agents like methicillin, oxacillin, cloxacillin, dicloxacillin, floxacillin, nafcillin; gram negative microorganism active agents like ampicillin, amoxicillin, and hetacillin, cillin, and galampicillin; antipseudomonal penicillins like carbenicillin, ticarcillin, azlocillin, mezlocillin, and piperacillin; cephalosporins like cefpodoxime, cefprozil, ceftbuten, ceftizoxime, ceftriaxone, cephalothin, cephapirin, cephalexin, cephradrine, cefoxitin, cefamandole, cefazolin, cephaloridine, cefaclor, cefadroxil, cephaloglycin, cefuroxime, ceforanide, cefotaxime, cefatrizine, cephacetrile, cefepime, cefixime, cefonicid, cefoperazone, cefotetan, cefinetazole, ceftazidime, loracarbef, and moxalactam, monobactams like aztreonam; and carbapenems such as imipenem, meropenem, pentamidine isethiouate, albuterol sulfate, lidocaine, metaproterenol sulfate, beclomethasone diprepionate, triamcinolone acetamide, budesonide acetonide, fluticasone, ipratropium bromide, flunisolide, cromolyn sodium, ergotamine tartrate and where applicable, analogues, agonists, antagonists, inhibitors, and pharmaceutically acceptable salt forms of the above. In reference to peptides and proteins, the invention is intended to encompass synthetic, native, glycosylated, unglycosylated, pegylated forms, and biologically active fragments and analogs thereof.

Active agents for use in the invention further include nucleic acids, as bare nucleic acid molecules, vectors, associated viral particles, plasmid DNA or RNA or other nucleic acid constructions of a type suitable for transfection or transformation of cells, i.e., suitable for gene therapy including antisense. Further, an active agent may comprise live attenuated or killed viruses suitable for use as vaccines. Other useful drugs include those listed within the Physician's Desk Reference (most recent edition).

The amount of active agent in the pharmaceutical formulation will be that amount necessary to deliver a therapeutically effective amount of the active agent per unit dose to achieve the desired result. In practice, this will vary widely depending upon the particular agent, its activity, the severity of the condition to be treated, the patient population, dosing requirements, and the desired therapeutic effect. The composition will generally contain anywhere from about 1% by weight to about 99% by weight active agent, typically from about 2% to about 95% by weight active agent, and more typically from about 5% to 85% by weight active agent, and will also depend upon the relative amounts of additives contained in the composition. The compositions of the invention are particularly useful for active agents that are delivered in doses of from 0.001 mg/day to 100 mg/day, preferably in doses from 0.01 mg/day to 75 mg/day, and more preferably in doses from 0.10 mg/day to 50 mg/day. It is to be understood that more than one active agent may be incorporated into the formulations described herein and that the use of the term “agent” in no way excludes the use of two or more such agents.

The pharmaceutical formulation may comprise a pharmaceutically acceptable excipient or carrier which may be taken into the lungs with no significant adverse toxicological effects to the subject, and particularly to the lungs of the subject. In addition to the active agent, a pharmaceutical formulation may optionally include one or more pharmaceutical excipients which are suitable for pulmonary administration. These excipients, if present, are generally present in the composition in amounts ranging from about 0.01% to about 95% percent by weight, preferably from about 0.5 to about 80%, and more preferably from about 1 to about 60% by weight. Preferably, such excipients will, in part, serve to further improve the features of the active agent composition, for example by providing more efficient and reproducible delivery of the active agent, improving the handling characteristics of powders, such as flowability and consistency, and/or facilitating manufacturing and filling of unit dosage forms. In particular, excipient materials can often function to further improve the physical and chemical stability of the active agent, minimize the residual moisture content and hinder moisture uptake, and to enhance particle size, degree of aggregation, particle surface properties, such as rugosity, ease of inhalation, and the targeting of particles to the lung. One or more excipients may also be provided to serve as bulking agents when it is desired to reduce the concentration of active agent in the formulation.

Pharmaceutical excipients and additives useful in the present pharmaceutical formulation include but are not limited to amino acids, peptides, proteins, non-biological polymers, biological polymers, carbohydrates, such as sugars, derivatized sugars such as alditols, aldonic acids, esterified sugars, and sugar polymers, which may be present singly or in combination. Suitable excipients are those provided in WO 96/32096, which is incorporated herein by reference in its entirety. The excipient may have a glass transition temperatures (Tg) above about 35° C., preferably above about 40° C., more preferably above 45° C., most preferably above about 55° C.

Exemplary protein excipients include albumins such as human serum albumin (HSA), recombinant human albumin (rHA), gelatin, casein, hemoglobin, and the like. Suitable amino acids (outside of the dileucyl-peptides of the invention), which may also function in a buffering capacity, include alanine, glycine, arginine, betaine, histidine, glutamic acid, aspartic acid, cysteine, lysine, leucine, isoleucine, valine, methionine, phenylalanine, aspartame, tyrosine, tryptophan, and the like. Preferred are amino acids and polypeptides that function as dispersing agents. Amino acids falling into this category include hydrophobic amino acids such as leucine, valine, isoleucine, tryptophan, alanine, methionine, phenylalanine, tyrosine, histidine, and proline. Dispersibility-enhancing peptide excipients include dimers, trimers, tetramers, and pentamers comprising one or more hydrophobic amino acid components such as those described above.

Carbohydrate excipients suitable for use in the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), pyranosyl sorbitol, myoinositol and the like.

The pharmaceutical formulation may also include a buffer or a pH adjusting agent, typically a salt prepared from an organic acid or base. Representative buffers include organic acid salts of citric acid, ascorbic acid, gluconic acid, carbonic acid, tartaric acid, succinic acid, acetic acid, or phthalic acid, Tris, tromethamine hydrochloride, or phosphate buffers.

The pharmaceutical formulation may also include polymeric excipients/additives, e.g., polyvinylpyrrolidones, derivatized celluloses such as hydroxymethylcellulose, hydroxyethylcellulose, and hydroxypropylmethylcellulose, Ficolls (a polymeric sugar), hydroxyethylstarch, dextrates (e.g., cyclodextrins, such as 2-hydroxypropyl-β-cyclodextrin and sulfobutylether-β-cyclodextrin), polyethylene glycols, and pectin.

The pharmaceutical formulation may further include flavoring agents, taste-masking agents, inorganic salts (for example sodium chloride), antimicrobial agents (for example benzalkonium chloride), sweeteners, antioxidants, antistatic agents, surfactants (for example polysorbates such as “TWEEN 20” and “TWEEN 80”), sorbitan esters, lipids (for example phospholipids such as lecithin and other phosphatidylcholines, phosphatidylethanolamines), fatty acids and fatty esters, steroids (for example cholesterol), and chelating agents (for example EDTA, zinc and other such suitable cations). Other pharmaceutical excipients and/or additives suitable for use in the compositions according to the invention are listed in “Remington: The Science & Practice of Pharmacy”, 19^{th} ed., Williams & Williams, (1995), and in the “Physician's Desk Reference”, 52^{nd} ed., Medical Economics, Montvale, N.J. (1998), both of which are incorporated herein by reference in their entireties.

“Mass median diameter” or “MMD” is a measure of mean particle size, since the powders of the invention are generally polydisperse (i.e., consist of a range of particle sizes). MMD values as reported herein are determined by centrifugal sedimentation, although any number of commonly employed techniques can be used for measuring mean particle size. “Mass median aerodynamic diameter” or “MMAD” is a measure of the aerodynamic size of a dispersed particle. The aerodynamic diameter is used to describe an aerosolized powder in terms of its settling behavior, and is the diameter of a unit density sphere having the same settling velocity, generally in air, as the particle. The aerodynamic diameter encompasses particle shape, density and physical size of a particle. As used herein, MMAD refers to the midpoint or median of the aerodynamic particle size distribution of an aerosolized powder determined by cascade impaction.

In one version, the powdered formulation for use in the present invention includes a dry powder having a particle size selected to permit penetration into the alveoli of the lungs, that is, preferably 10 μm mass median diameter (MMD), preferably less than 7.5 μm, and most preferably less than 5 μm, and usually being in the range of 0.1 μm to 5 μm in diameter. The delivered dose efficiency (DDE) of these powders may be greater than 30%, more preferably greater than 40%, more preferably greater than 50% and most preferably greater than 60% and the aerosol particle size distribution is about 1.0-5.0 μm mass median aerodynamic diameter (MMAD), usually 1.5-4.5 μm MMAD and preferably 1.5-4.0 μm MMAD. These dry powders have a moisture content below about 10% by weight, usually below about 5% by weight, and preferably below about 3% by weight. Such powders are described in WO 95/24183, WO 96/32149, WO 99/16419, and WO 99/16422, all of which are all incorporated herein by reference in their entireties.

Although the present invention has been described in considerable detail with regard to certain preferred versions thereof, other versions are possible, and alterations, permutations and equivalents of the version shown will become apparent to those skilled in the art upon a reading of the specification and study of the drawings. For example, the cooperating components may be reversed or provided in additional or fewer number. Also, the various features of the versions herein can be combined in various ways to provide additional versions of the present invention. Furthermore, certain terminology has been used for the purposes of descriptive clarity, and not to limit the present invention. Therefore, any appended claims should not be limited to the description of the preferred versions contained herein and should include all such alterations, permutations, and equivalents as fall within the true spirit and scope of the present invention.

1. A pharmaceutical package comprising:
a first compartment containing an aerosolization apparatus, the aerosolization apparatus comprising:
a housing defining a chamber having one or more air inlets and one or more air outlets, the chamber being sized to contain a capsule;
a puncturing mechanism moveable within the housing to create one or more openings in a capsule in the chamber;
a mouth or nose piece in communication with the one or more air outlets, whereby when a user inhales, air enters into the chamber through the inlet to cause the capsule to move within the chamber so that the pharmaceutical formulation exits through one or more openings in the capsule and is aerosolized and transported through the outlet for delivery to the user through the mouth or nose piece; and the pharmaceutical package further comprising
a second compartment containing a capsule, the capsule being positionable within the chamber of the aerosolization apparatus during use of the aerosolization apparatus,
wherein the capsule contains an aerosolizable powder pharmaceutical formulation comprising amphotericin B, wherein the capsule is contained within the second compartment by a moisture barrier seal.

2. A pharmaceutical package according to claim 1 wherein the capsule contains a pharmaceutical formulation comprising at least 5 mg of amphotericin B.

3. A pharmaceutical package according to claim 1 wherein the capsule contains a pharmaceutical formulation comprising a lipid.

4. A pharmaceutical package according to claim 1 wherein the capsule contains a pharmaceutical formulation comprising particles having a mass median diameter less than 20 μm.

5. A pharmaceutical package according to claim 1 wherein the capsule contains a pharmaceutical formulation comprising particles having a mass median diameter less than 10 μm.

6. A pharmaceutical package according to claim 1 wherein the second compartment contains a plurality of capsules, the capsules together making up a dose of amphotericin B to be administered.

7. A pharmaceutical package according to claim 1 comprising a plurality of second compartments.

8. A pharmaceutical package according to claim 7 wherein each of the second compartments are separately sealed.

9. A pharmaceutical package according to claim 1 wherein the one or more air inlets in the aerosolization apparatus are shaped to create a swirling flow in the chamber.

10. A pharmaceutical package according to claim 9 wherein the moisture barrier seal comprises a metal foil.

11. A pharmaceutical package according to claim 9 wherein the moisture barrier seal comprises two layers of a metal foil with the capsule being contained between the layers.

12. A pharmaceutical package according to claim 1 wherein the aerosolization apparatus is contained within the first compartment by a moisture barrier seal.
